(19) United States
(12) Patent Application Publication
(10) Pub. No.: US 2026/0069801 A1
(43) Pub. Date: Mar. 12, 2026

(54) MEDICAL DEVICE WITH ALARM COORDINATION

(71) Applicant: Drägerwerk AG & Co. KGaA, Lübeck (DE)

(72) Inventors: Thomas KRÜGER, Lübeck (DE); Birger LANDWEHR, Lübeck (DE)

(21) Appl. No.: 19/324,276

(22) Filed: Sep. 10, 2025

(30) Foreign Application Priority Data
Sep. 12, 2024 (DE) 10 2024 126 208.7

(51) Int. Cl. A61M 16/00

(52) U.S. Cl. CPC A61M 16/0051; A61M 16/0003; A61M 16/024; A61M 2016/0033; A61M 2205/18; A61M 2205/3386

(57) ABSTRACT

A medical device for ventilating a living being has functions for coordinating alarms (60, 60′). The configuration of the coordination is aimed at detecting undershooting (31) of a predetermined lower threshold value (30) of a tidal volume VT (13, 13′). The configuration makes it possible to avoid overestimations of non-critical situations (10′) by means of alarm delays, but is sufficiently sensitive with regard to critical situations (10).

### CROSS REFERENCE TO RELATED APPLICATIONS

This application claims the benefit of priority under 35 U.S.C. § 119 of German Application 10 2024 126 208.7, filed Sep. 12, 2024, the entire contents of which are incorporated herein by reference.

### TECHNICAL FIELD

The present invention relates to a medical device with alarm coordination, in particular a medical device suitable for ventilating a living being or a patient, such as an anesthesia device, an intensive care ventilator, a home ventilator, a mobile emergency ventilator or ventilators for ventilating premature and/or newborn babies, infants and children.

### BACKGROUND

During ventilation, certain ventilation parameters such as ventilation pressures, ventilation frequency (RR), tidal volume (VT), I:E ratio, minute volume (MV) are set and monitored by sensors. If, in the course of such monitoring, events or situations arise with deviations from predefined lower and/or upper comparison values or threshold values, an alarm management system of the medical device typically intervenes to inform the user of the events or situations. A key parameter that is of great interest to monitor during ventilation is the tidal volume VT. The tidal volume is the amount of breathing gas (respiratory gas) mixture that is set individually for each patient in order to supply the lungs of this patient with a sufficient amount of breathing gas mixture. A setting with a sufficiently large tidal volume is important to ensure effective gas exchange. However, the tidal volume must not be too large in order to avoid overstretching the lungs during ventilation. Therefore, both the setting of the tidal volume and the alarm thresholds for the tidal volume by the healthcare professional on the medical device and the sensory monitoring of the alarm thresholds for the tidal volume by the medical device ensure a very important aspect of high-quality ventilation therapy for maintaining the health of the patient or for recovery. A value in a range of 80% to 90% of the set target tidal volume VT_Set is often selected as a lower alarm threshold VT_Low.

In the prior art, it is possible to send alarms to a user depending on criteria or depending on certain situations.

For example, U.S. Pat. Nos. 9,962,509 and 9,615,743 show some possibilities for reducing ventilation alarms, in particular reducing alarms for the minute volume (MV). So-called soft-threshold methods for reducing alarms with alarm threshold values adjusted during operation are also known, as described, for example, in U.S. Pat. No. 6,754,516, EP2302606 B1, EP2245607 B1 or U.S. Pat. No. 8,515,513.

### SUMMARY

It is an object of the invention to specify and provide an alarm coordination for a medical device.

The task of specifying an alarm coordination for a medical device is solved by a medical device with features according to this disclosure.

Advantageous embodiments of the invention result from and are explained in more detail in the following claims, description with partial reference to the figures.

According to the invention, a medical device for ventilating a living being or a patient with alarm coordination is configured for a robust implementation and configuration of an alarm which is directed to one or more undershoots of (occasions of falling below) a predetermined (preset/specified) lower volume threshold value of a tidal volume. On the one hand, the robust configuration makes it possible to avoid overestimating non-critical situations. On the other hand, however, the robust configuration is sufficiently sensitive with regard to critical situations.

A basic idea of the invention is to provide an alarm delay time between the detection of a tidal volume falling below a lower volume threshold value and the actual activation of the alarm (the issuance of an alarm) to the user based on this, which alarm delay time is dependent on a difference between the current value indicating a tidal volume and the lower volume threshold value of the tidal volume.

A control unit is suitably configured and provided to implement the basic idea of alarm coordination described above in a medical device configured to ventilate a living being or a patient.

The control unit is configured to control mechanical ventilation, i.e., for example, to control pressure-controlled ventilation or volume-controlled ventilation. The control unit can also be configured to control and/or coordinate supportive forms of ventilation on the basis of measured values of ventilation pressures, inspiratory and/or expiratory flow rates or volumes.

Furthermore, the control unit is configured to control and/or manage alarm functions of the ventilator, including in particular threshold value monitoring of inspiratory and/or expiratory ventilation pressures, threshold value monitoring of carbon dioxide concentrations, threshold value monitoring of oxygen concentrations, threshold value monitoring of inspiratory and/or expiratory flow rates.

The control unit is configured to coordinate the robust implementation and configuration of an alarm according to the invention, which is directed to one or more undershoots of a predetermined lower threshold value of a tidal volume.

A medical device according to the invention is configured for ventilating a living being and comprises the control unit, a breathing gas supply system, a line system (breathing gas conduit system/breathing gas guide system) and a sensor system.

The breathing gas supply system has a gas conveying unit and a dosing unit for controlled dosing of an inspiratory breathing gas quantity.

The breathing gas supply system has at least one device for controlling the expiratory breathing gases. For example, an expiratory valve (PEEP valve-positive end expiratory pressure valve) is usually provided to control the airway pressure in the breathing gas supply system

The line system is configured to supply breathing gas quantities to the living being and to guide breathing gas quantities away from the living being.

The sensor system has at least one flow rate sensor, which is arranged on the breathing gas supply system or on the line system in such a way as to continuously detect at least one measured value of a flow rate and provide it to the control unit as measured values.

The control unit is configured to carry out and coordinate a controlled dosing of the inspiratory breathing gas quantity for ventilation of the living being with the sensor system, the breathing gas supply system and the line system.

The control unit is configured to determine a tidal volume VT on the basis of the measured values of the flow rate sensors.

The control unit is configured to coordinate alarms during the course of ventilation over a plurality of inspiratory dosing periods, with the alarms being directed to undershoots of a lower volume threshold value of the tidal volume.

The control unit is further configured to consider a degree of undershoot (a shortfall level or undershoot indication) as a first criterion for the coordination of alarms.

The degree of undershoot indicates the degree to which a tidal volume falls below the lower volume threshold.

The control unit is configured to coordinate the alarms by adjusting an alarm delay as a function of the degree of undershoot. The adjustment of the alarm delay is based on the degree of undershoot being one criterion for adjustment, with adjustment of the alarm delay preferably being based on other criteria as well, as discussed herein.

The control unit uses the first criterion of the degree of undershoot to determine how the situation develops during ventilation with regard to a desired tidal volume applied to the living being.

By continuously determining the degree to which the tidal volume falls below the lower volume threshold, a form of trend analysis or trend tracking for the tidal volume is provided.

The alarm delay can be selected by the control unit as an alarm delay time depending on the degree of undershoot of the lower volume threshold. Slight undershooting of the lower volume threshold value leads to longer alarm delay times, while significant undershooting of the lower volume threshold value leads to shorter alarm delay times down to an alarm delay time of 0.00 seconds.

The lower volume threshold value can be specified by the user or derived from the VT_set tidal volume setting value. The lower volume threshold for an alarm can be set by the user, for example, to 90% of the desired tidal volume to be applied to the living being.

Alternatively, the lower volume threshold value can also be directly linked to the setting value of the tidal volume VT_set; this is a question of the design in practice with scaling of the threshold values when implementing the coordination of alarms with alarm delays in the respective setup of a ventilator.

Here is an example with an assignment that can be selected by the control unit when coordinating alarms in relation to the tidal volume to select a suitable alarm delay time. The alarm delay can be configured as an alarm delay time for an adult patient (male, 80 kg body weight), for example as case a):
90 seconds if the volume falls below the lower volume threshold by less than 10%,
60 seconds if the volume falls below the lower volume threshold by approximately 50%,
30 seconds if the volume falls below the lower volume threshold by more than 90%

The alarm delay can be configured as an alarm delay time for an infant or a small child (4.5 kg to 8 kg body weight), for example as case b):
60 seconds if the volume falls below the lower volume threshold by less than 10%,
30 seconds if the volume falls below the lower volume threshold by approximately 50%,
10 seconds if the volume falls below the lower volume threshold by more than 90%

Alternatively or in addition to the configuration of the delays with a time reference of predetermined periods—in the above example 0s, 30s, 60s, 90s—depending on the degree to which the volume falls below the lower volume threshold value, the time reference can also be derived from the breathing frequency or the ventilation frequency by selecting the alarm delay depending on the degree to which the volume falls below (undershoots) the lower volume threshold value for a predetermined number of breathing cycles (respiratory cycles) or ventilation cycles. Slight undershooting of the lower volume threshold leads to an alarm delay of several breathing cycles. Significant undershooting of the lower volume threshold leads to alarm delays of a few breathing cycles.

Such an alarm delay for an infant or a small child (4.5 kg to 8 kg body weight; case b) can be structured as follows as an example:
50 breathing cycles if the volume falls below the lower volume threshold by less than 10%,
25 breathing cycles if the volume falls below the lower volume threshold by more than 50%.
8 breathing cycles if the volume falls below the lower volume threshold by more than 90%

The configuration of the alarm delay as a function of the degree of undershoot of the lower volume threshold for a predetermined number of breathing cycles or ventilation cycles makes it possible to scale the respiratory rate (RR) with the number of breathing cycles predetermined for an alarm delay for each patient type (premature and newborn babies, children, adults) to produce a period of the alarm delay that is individually adapted to the situation. Here is an example for case b): Newborn child, 2.5 kg body weight, tidal volume VT of 0.015 liters, RR=50 min⁻¹.

If the set tidal volume VT_set is undershot by 10%, the following situation arises in case b):
predetermined period of the alarm delay of 60 seconds
delay by a number of 50 breathing cycles until the alarm is triggered, resulting in a period of 60 seconds until the alarm delay with RR=50 min⁻¹

The options described above show possible configurations of implementations based on threshold values, preferably threshold values in steps. Instead of an implementation based on threshold values and comparison operations (<. <, =) with threshold values, an implementation can also be carried out using a data table (look up table). In this case, an assignment of associated periods of alarm delays or an assignment of an associated number of breathing cycles as a configuration of an alarm delay can be stored in the form of data sets (data records) for different degrees of undershooting the lower volume threshold value.

Such a data table can, for example, be configured with a step width (size) in 0.5% increments of undershooting of the lower volume threshold value, so that a fine-grained configuration can be created for the alarm delay. The fine-grained configuration in the data table can be based on a linear, continuous relationship, a discontinuous relationship, a non-linear and progressive continuous relationship or combinations of linear, non-linear, progressive as well as continuous and discontinuous relationships. As an alternative to using a data table (look up table), a general functional relationship can also be selected.

A significant advantageous effect of the coordination of alarms by means of predetermined and defined alarm delays is that occasional minor undershooting of the tidal volume below the lower volume threshold value does not cause alarms in relation to the alarm situation VT_Low of a tidal volume that is too low.

A further advantageous effect is that only persistent undershooting of the tidal volume below the lower volume threshold value causes an alarm in relation to the alarm situation VT_Low of a tidal volume that is too low.

In a preferred embodiment, the control unit can be configured to also consider a further criterion for the coordination of alarms. A frequency indicator can serve as a further criterion; the frequency indicator can indicate how often during a predetermined number of time periods with inspiratory doses the tidal volumes (undershoots) are below the lower volume threshold.

The inclusion of the frequency indicator makes it possible in an advantageous way that even permanently persistent smaller undershooting of the tidal volume below the lower volume threshold value causes an alarm in relation to the alarm situation VT_Low of a tidal volume that is too low.

In a further preferred embodiment, the control unit can be configured to carry out averaging or mean value calculation of tidal volumes over at least two inspiratory phases. Options for averaging or mean value calculations can be configured using various functions such as arithmetic, geometric, harmonic or quadratic averaging, moving average, median filter, ranking filter. An advantageous effect of this embodiment with averaging is that no alarms are triggered in relation to the VT_Low alarm situation of an insufficient tidal volume for situations in which the volume falls significantly below the lower volume threshold, a situation which only occurs very rarely over the course of ventilation, for example once during a sequence of more than 50 breaths. Such infrequent undershoots can occur, for example, due to random and irregular disturbances in the pneumatic coupling of patient and ventilator via the ventilation circuit and a nasal mask and are detected in normal operation by routines and alarms designed for this purpose and reported to the user, so that by coordinating the alarms with regard to the alarm situation VT_Low of an insufficient tidal volume according to this preferred embodiment with averaging over multiple phases, multiple alarms can be reduced.

For practical implementation, it may be advantageous to adjust the period of the alarm delay with the averaging with the number of measured values used for averaging and/or the sampling rate of the measured values of the flow rate sensors.

For practical implementation, it can also be advantageous to adjust the period of the alarm delays to the type and method of ventilation used, such as ventilation modes with volume or pressure control, and/or to adjust them to ventilation parameters such as ventilation frequency (RR), inspiration time period (T_i), expiration time period (T_e).

This results in further preferred embodiments with a configuration of the control unit in which the averaging or filtering can be adjusted to the period of the alarm delay and/or the configuration of the averaging or filtering can be coordinated with the type and manner of ventilation used.

Thus, in a further preferred embodiment, the control unit can be configured to include a type of ventilation mode used during ventilation, in particular volume-controlled or pressure-controlled ventilation modes, in the coordination of alarms. Inclusion takes place, for example, in such a way that a different adjustment of the lower volume threshold value or degree of undershoot and/or the frequency indicator is carried out by the control unit during operation with regard to volume-controlled ventilation or pressure-controlled ventilation and is advantageously integrated into the alarm management of the ventilator.

In a further preferred embodiment, the control unit can be a configured to use averaging or mean value calculation or methods of interpolation, in particular by means of linear or non-linear interpolation, when determining the degree of undershoot and/or frequency indicator. An advantageous effect results from embodiments with interpolation in a similar way to the already mentioned embodiments with averaging. Frequent alarms in relation to the alarm situation VT_Low of a tidal volume that is too low as a result of one-time significant undershooting of the lower volume threshold value are reduced by interpolation and/or averaging.

In a further preferred embodiment, the control unit can be configured to carry out data processing with a maximum upper threshold of the determined current tidal volume VT to a maximum value before or during the execution of averaging and/or interpolation over the tidal volume. For example, a limitation to a maximum upper value of 110% of the target value of the tidal volume can be carried out. The upper threshold has the advantage that individual, significantly excessive values during subsequent averaging do not lead to a distortion of the tidal volumes actually physiologically effective in the patient's lungs during ventilation.

In a further preferred embodiment, a data memory can be assigned to the control unit. The data memory is configured to store information on the degree of undershoot and/or the frequency indicator in the form of data sets or tables with threshold values.
threshold values
tolerance bands, comparison values, comparison curves
default values, preset values
predetermined alarm delays
   or in the form of data sets or tables with predetermined alarm delay times corresponding to the degree of undershoot and/or the frequency indicator
predetermined alarm delay periods
predetermined numbers of breathing cycles until alarming (an alarm is issued) and make them available to the control unit.

The data memory can be configured as part of the control unit or as independent units connected to the control unit in data exchange.

For this purpose, the control unit can, for example read in a lower volume threshold value from the data memory, which is to be used for ventilating a newborn. The control unit can, for example, read in a predetermined alarm delay time of 60 seconds or 50 breathing cycles corresponding to a specific degree of undershoot of, for example, 90%, which is to be used for ventilating a newborn.

In a further preferred embodiment, the control unit can be configured to include patient demographic criteria in the coordination of alarms.

Patient demographic criteria are, for example, criteria that differentiate between patient types such as premature babies, newborns, infants, toddlers, children, adolescents and adults on the basis of age. For example, the typical number of breathing cycles until the alarm is triggered or the alarm delay time for adults can be significantly longer than for infants, as the larger blood volume in the bloodstream of an adult compared to the blood volume in the bloodstream of an infant means that a possible undersupply of oxygen to areas of the brain does not occur until much later.

In a further preferred embodiment, the control unit can be configured to include patient-specific criteria such as gender, weight, height, information on pathological situations, information on diagnostics, therapy as well as information on medication, medical devices or implants when coordinating alarms.

In a further preferred embodiment, the control unit can be configured to include information or a value that indicates an oxygen saturation in the blood of the living being or the patient (e.g. SPO₂) when coordinating alarms.

In a further preferred embodiment, the control unit can be configured to include information or a value which indicates an expiratory carbon dioxide concentration, in particular an end-expiratory carbon dioxide concentration (etCO₂) in the breathing gas of the living being or the patient, in the coordination of alarms.

In a further preferred embodiment, the control unit can be configured to include information indicating a ratio of the oxygen saturation to the expiratory carbon dioxide concentration in the coordination of alarms.

Information on oxygen saturation and/or expiratory carbon dioxide concentration as well as information on the ratio of oxygen saturation to expiratory carbon dioxide concentration during alarm coordination indicates whether the gas exchange in the patient's lungs may be disturbed when oxygen is absorbed into the patient's bloodstream and/or whether the patient's ability to exhale carbon dioxide may be disturbed. In such situations, the alarm coordination can be adapted by the control unit, for example to deactivate any alarm delays.

In summary, the present invention makes it possible to coordinate the alarms in relation to a supply of too low a tidal volume in a suitable patient-specific manner in such a way that situations with slight undershoots and, in particular, situations with individual slight undershoots of the lower volume threshold value by the currently supplied tidal volume do not immediately lead to visual and/or acoustic alarms. Instead, the alarm delay ensures that the user is only alerted (an alarm is only issued) if the tidal volume currently being supplied falls below the lower volume threshold value for a longer period of time.

In the following, exemplary embodiments of the invention are explained in more detail with reference to the figures, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a graph showing a situation and an alarm situation during ventilation;

FIG. 2 is a graph showing another situation and another alarm situation during ventilation;

FIG. 3 is a graph showing time courses with breathing cycles, tidal volumes, and alarm situations; and

FIG. 4 is a schematic view showing the structure of a ventilator.

### DESCRIPTION OF PREFERRED EMBODIMENTS

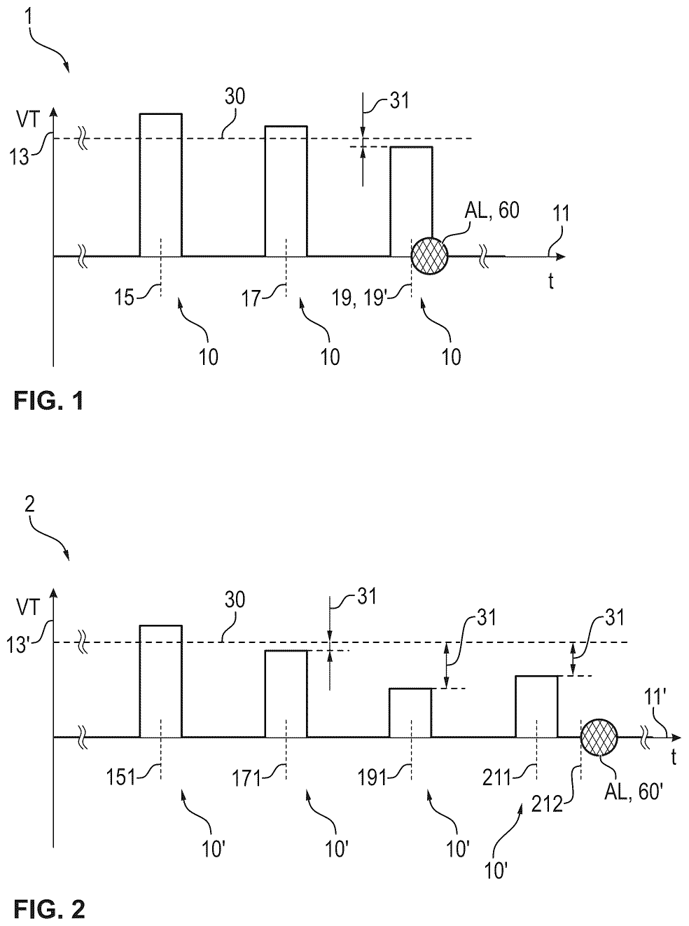

Referring to the drawings, identical elements in FIGS. 1, 2, 3, 4 are labeled with identical reference numbers in FIGS. 1, 2, 3, 4.

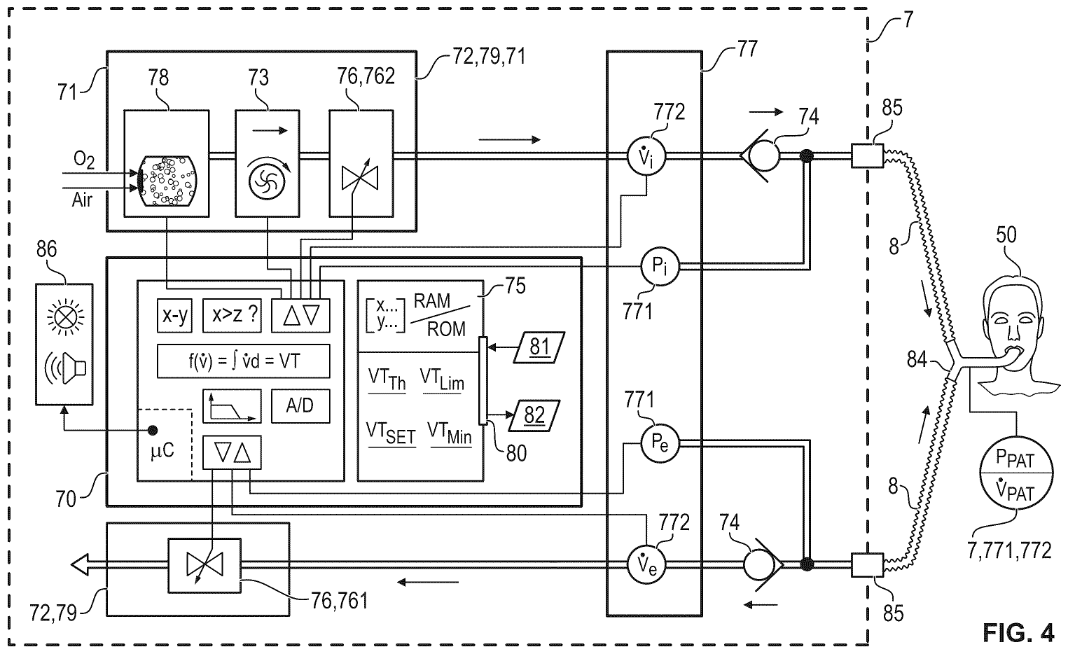

FIG. 1 and FIG. 2 show two different situations 10, 10′ during ventilation of a living being 50 (FIG. 4) with regard to the course of the tidal volume VT, which are explained in more detail below in a joint description. FIG. 1 shows in a representation 1 a time course 11 with a representation of a number of tidal volumes 13 recorded at points in time 15, 17, 19 by a measuring technique in a ventilator. FIG. 2 shows in a representation 2 a time course 11′ with a representation of a number of tidal volumes 13′ recorded at points in time 151, 171, 191, 211. FIGS. 1 and 2 can be used to illustrate two basic scenarios for alerting a user to the situations 10, 10′. The tidal volumes at the times 15, 17 (FIG. 1) are above a volume threshold value 30. The tidal volume at the time 19 (FIG. 1) is below the volume threshold value 30. The tidal volume at the time 151 (FIG. 2) is above the volume threshold value 30. The tidal volumes at the times 171, 191, 211 (FIG. 2) are below a volume threshold value 30. The deviations of the tidal volumes below the volume threshold values 30 are shown in FIGS. 1 and 2 as undershoots 31.

With a criterion that is aimed towards an immediate alarm to the user each time there is an undershot 31 of the volume threshold value 30, an immediate alarm AL 60 would be sent to the user without a delay at a time 19′. as can be seen from FIG. 1.

In the case of a criterion that is aimed at alerting the user when the time there is an undershooting 31 of the volume threshold value 30 several times, the alert AL 60′ would be sent to the user at time 212.

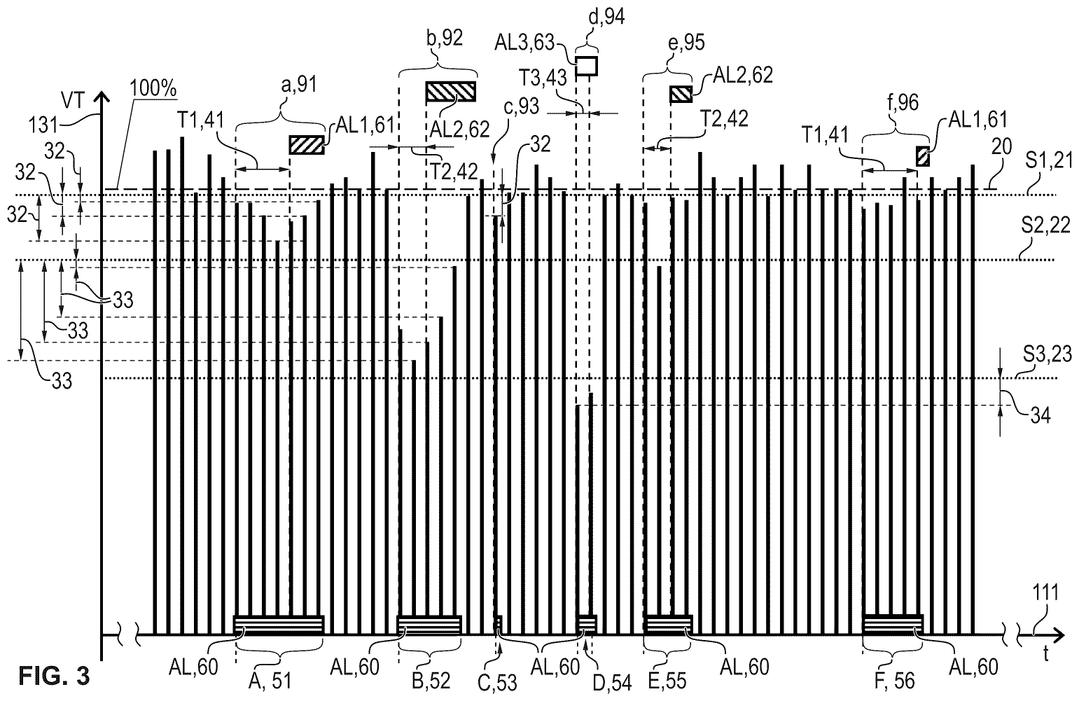

FIG. 3 shows a time course 111 (X-axis) with a representation (Y-axis) of a plurality of tidal volumes Vt 131 over approximately 60 breathing cycles as bars in a bar diagram with representation of a reference value 20 (dashed line) of 100% of the tidal volume VT 131 and representation of three threshold values S1 21, S2 22, S3 23 (dotted line) related to this reference value 20. The threshold values S1 21, S2 22, S3 23 are shown in this FIG. 3 by way of example as three different threshold values in relation to the reference value 20.

The situations A, B, C, D, E, F, as they would result according to FIGS. 1 and 2 with regard to an alarm 60 to the user without taking into account a degree of undershoot of the current tidal volume below the reference value 20 of 100%, are shown near the X-axis 111 in the lower area of FIG. 3.

The situations a, b, c, d, e, f, as they arise in practice when applying the alarm coordination in the sense of an implementation of the present invention with coordinated alarming 61, 62, 63 to the user, are shown close to the representation of the reference value 20 in the upper region of FIG. 3. It should be noted that the time course in FIG. 3 represents an exemplary compilation and random sequence of alarm situations and does not necessarily correspond to the course of a real ventilation of a living being. This applies both to the respective period of the different alarm situations and to the sequence of the different alarm situations with only short time intervals between the different alarm situations. The time course 111 in this figure is thus merely fictitious and arbitrarily chosen in order to be able to graphically and schematically represent a graphic, schematic representation for visualizing the present invention of alarm coordination for various situations A, B, C, D, E, F, 51, 52, 53, 54, 55, 56 or a, b, c, d, e, f 91, 92, 93, 94, 95, 96 in a common representation.

An essential feature of the present invention of alarm coordination is, as already explained in the general part of the description, that the period of the delay until the alarm is sent to the user is determined as a function of the degree to which a value of a current tidal volume VT 131 undershoots (falls below) the reference value 20.

In FIG. 3, three different periods T1 41, T2 42, T3 43 of delays up to alarms AL1 61, AL2 62, AL3 63 to the user are shown for a simplified illustration of the three threshold values S1 21, S2 22, S3 23 related to the reference value 20. For the various situations A, B, C, D, E, F, 51, 52, 53, 54, 55, 56, alarms AL 60 would be triggered without the use of alarm coordination. With the application of alarm coordination, alarms AL1 61, AL2 62, AL3 63 are then triggered for situations a, b, c, d, e, f, 91, 92, 93, 94, 95, 96, delayed by the time periods T1 41, T2 42, T3 43. Alarms are triggered in situations a 91, b 92, d 94, e 95, f 96. No alarms are triggered in situation c 93. The alarms AL 60 and AL1 61, AL2 62, AL3 63 are shown as rectangular bars with different types of hatching.

Situations A, B, C, D, E, F, 51, 52, 53, 54, 55, 56 or a, b, c, d, e, f, 91, 92, 93, 94, 95, 96 are now used to explain the mode of operation of the alarm coordination.

If the current tidal volume VT 131 falls only slightly below the reference value 20, i.e. below the threshold value S1 21 and the current tidal volume VT 131 is above the threshold value S2 22, a time delay with a first time period T1 41 is waited for before an alarm A1 61 is issued to the user. Such minor undershoots 32 are given in the situations a 91 and f 96.

If the current tidal volume VT 131 falls below the reference value 20, i.e. below the threshold value S2 22 and the value of the current tidal volume VT 131 is above the threshold value S3 23, a time delay with a second time period T2 42 is waited for before an alarm A2 62 is issued to the user. Such undershoots 33 are given in situations b 92 and e 95.

If the current tidal volume VT 131 falls significantly below the reference value 20, i.e. below the threshold value S3 23, a time delay with a third time period T3 43 is waited for before an alarm A3 63 is issued to the user. In FIG. 3, for example, the time delay of the third time period T3 43 is configured as an immediate and direct alarm—i.e. with a third time period of T3 43 of almost 0.0 seconds. Such an undershoot 34 is given in situation d 94.

In situation c 93, the slight undershot 32 is only for a very short time, i.e., for a period below the period of the first time period T1 41—so in situation c 93 no alarm is activated, in contrast to situation C 53. In situation f 96, additionally to the undershoots at the beginning of the first time period T1 41 the current tidal volume VT 131 falls below the threshold value S1 21 at the end of the first time period T1 41, too. So, the alarm AL1 61 is activated.

In the context of the present invention, for ventilation, the possible periods T1 41, T2 42, T3 43 of an alarm delay in a range of 10 to 60 seconds are suitable for implementation during the ventilation.

A criterion related to a frequency indicator may be implemented in a way that during a long moving time interval of dosed tidal volumes the number of tidal volumes below the second threshold S2 is counted and a ratio [%] related to the total amount of tidal volumes within this time interval is calculated. The long time interval may have three times the duration of full scale x-axis of FIG. 3. This may be in a range of 5 minutes. In case that during this interval a certain percentage of tidal volumes below the second threshold S2, for example 15%, has occurred, an alarm will be issued.

With regard to the assignment of degrees of undershooting the reference value 20 of the tidal volume VT 131 and the alarm delays Cycledelay and Tdelay derived therefrom in the context of the present invention, reference is made to the explanations and listings in the general part of the description.

FIG. 4 shows a schematic representation of a medical device configured as a ventilator 7 for ventilating a living being or patient 50, which is configured to form an alarm coordination system. The ventilator 7 and patient 50 are connected via connection elements 85 to a line system 8 configured as a breathing tube system with an inspiratory breathing tube for supplying fresh inhaled gases to the patient 50 and with an expiratory breathing tube for carrying exhaled gas away from the patient 50. The ventilator 7 has a sensor system 77 with a pressure sensor system 771 and a flow rate sensor system 772 as well as a dosing unit 71 with a gas mixing unit 78 for providing a breathing gas mixture with defined proportions of air (Air) and oxygen (O₂) and a gas conveying unit 73. A control unit 70 with an associated data memory 75 is provided to coordinate and monitor the ventilation of patient 50 and to coordinate alarms. The control unit 70 is equipped with a computing unit (μC), driver stages, signal processing such as amplifiers (OP amps), filter circuits, A/D converters and an associated data memory 75 and is configured to control the actuators 71, 72, 73, 76, 78, 79, 761 762 during ventilation. To carry out the ventilation and also to carry out the alarm coordination, measured values recorded and provided by the sensors 77, 771, 772 are used by the control unit 70 in combination with criteria such as, in particular, threshold values and default values.

In a suitably designed configuration, the control unit 70 has the necessary functions and/or program code (one or more programs) for carrying out mathematical or logical operations such as difference formation (subtraction), integral calculation or for carrying out comparisons with reference values, threshold values or tolerance bands in order to be able to configure the alarm coordination as well as the ventilation control. Inputs 81 of data or information can be made to the control unit 70 via an interface 80 and/or outputs 82 of data or information can be provided externally by the control unit 70.

The data memory 75 is configured to store, hold and provide information in relation to threshold values, in particular volume threshold values 30 (FIG. 1, FIG. 2), information in relation to criteria such as a degree of undershoot 31 (FIG. 1, FIG. 2) and or a frequency indicator.

The pressure sensor system 771 as well as the flow rate sensor system 772 can be configured for inspiratory, expiratory and/or patient proximal use on a connecting element 84 of the breathing tube system 8, the so-called “Y-piece”. The sensor system 77 continuously provides measured values to the control unit 70 to coordinate ventilation and alarms. The tidal volumes supplied to the patient 50 during ventilation in the respective breathing cycle are determined by the control unit 70 from the measured values of the flow rate sensors by means of time integration over time. In addition, a valve unit 79 with actively controllable valves 76, for example in the form of an expiratory valve 761 and an inspiratory dosing valve 762, is provided by the control unit 70 in order to effect the dosing of gas volumes to the patient 50 and, in conjunction with passive non-return valves 74, to determine the direction of the gas flows in the ventilation tube system 8. The valve unit 79 and the dosing unit 71 together form a breathing gas supply system 72. An alarm unit 86 is connected to the control unit, which is configured to provide alarms and/or signaling to a user in an acoustic and/or visual manner, for example also coordinated alarms with predetermined alarm delays to the user if a volume threshold value 30 (FIG. 1, FIG. 2) is undershot using the at least one criterion of a degree of undershoot.

While specific embodiments of the invention have been shown and described in detail to illustrate the application of the principles of the invention, it will be understood that the invention may be embodied otherwise without departing from such principles.

### REFERENCE NUMBER LIST

1, 2 Situation Representation
7 Ventilator, medical device
8 Ventilation tube system, line system
10, 10′ Situations, events
11, 11′, 111 Time courses, time axis t, X-axis
13, 13′, 131 Tidal volume, Y-axis
17, 19, 19′, 21, 151, 171, 191, 211, 212 Time points
20 Reference value
21, 23, 23, 30 Volume thresholds, alarm threshold
31, 32, 33, 34 Undershooting of volume thresholds
41, 42, 43 Time delays T1, T2, T3
50 Patient, living being
51, 52, 53, 54, 55, 56 Situations A, B, C, D, E, F
60, 60′, 61, 62, 63 Alerts
70 Control unit, μC
71 Dosing unit
72 Breathing gas supply system
73 Gas conveying unit (blower)
74 Non-return valves (check valves), passive valves
75 Data memory (RAM, ROM)
76, 761, 762 Valves, expiratory valve, inspiratory dosing valve
77, 771, 772 Sensors, pressure sensors, flow rate sensors
78 Gas mixing unit
79 Valve unit
80 Interface, interface
81 Inputs, input
82 Outputs, output
84 Connecting element (Y-piece)
85 Connecting elements
86 Alarm unit
91, 92, 93, 94, 95, 96 Situations a, b, c, d, e, f

What is claimed is:

1. A medical device with alarm coordination for ventilation of a living being, the medical device comprising:
a control unit;
a breathing gas supply system, which comprises a gas conveying unit, a dosing unit for controlled dosing of an inspiratory breathing gas quantity and a device for controlling an expiratory breathing gas quantity;
a sensor system, which comprises a flow rate sensor, which is arranged in operative connection with the breathing gas supply system to continuously detect at least one measured value of a flow rate and to make the measured value available to the control unit as measured values; and
a line system, which is configured to supply quantities of breathing gas to the living being and to guide quantities of breathing gas away from the living being,
wherein the control unit is configured to carry out and coordinate with the sensor system, the breathing gas supply system and the line system, a controlled dosing of the inspiratory breathing gas quantity for ventilation of the living being,
wherein the control unit is configured to determine a tidal volume based on the measured values of the flow rate sensors,
wherein the control unit is configured to carry out a coordination of alarms during a course of ventilation over a plurality of inspiratory dosing periods, the alarms being directed to undershoots of a lower volume threshold value of the tidal volume,
wherein the control unit is configured to determine a degree of undershoot as a first criterion for the coordination of alarms, which degree of undershoot indicates a degree to which a tidal volume falls below the lower volume threshold value,
wherein the control unit is configured to carry out an adjustment of an alarm delay as a function of the degree of undershoot.

2. A medical device according to claim 1, wherein the control unit is configured to determine a frequency indicator as a further criterion for the coordination of alarms, wherein the frequency indicator indicates a frequency with which a current tidal volume falls below the lower volume threshold value during a predetermined number of periods with inspiratory dosages and the control unit is configured to carry out the adjustment of the alarm delay based also on the frequency indicator.

3. A medical device according to claim 1, wherein the control unit is configured to carry out averaging, mean value calculations or filtering over tidal volumes over at least two inspiratory phases.

4. A medical device according to claim 2, wherein the control unit is configured to determine the degree of undershoot and/or the frequency indicator based on an averaging or a mean value calculation or a method of linear interpolation or a method of non-linear interpolation.

5. A medical device according to claim 4, wherein the control unit is configured to implement an upper threshold of the determined current tidal volume before or during the averaging and/or interpolation over the tidal volume.

6. A medical device according to claim 2, further comprising a data memory that is operatively connected with the control unit, wherein the data memory is configured to store, save and make available to the control unit the degree of undershoot and/or the frequency indicator as one or more of:
tables or tables with threshold values or tolerances;
data sets or tables with threshold values or tolerance bands;
data sets or tables with comparison values and/or comparison curves; and
data sets or tables with specifications and/or specification curves.

7. A medical device according to claim 1, wherein the control unit is configured to include in the coordination of alarms a type of ventilation mode used during ventilation of the living being.

8. A medical device according to claim 1, wherein the control unit is configured to include patient-demographic criteria or patient-specific criteria in the coordination of alarms.

9. A medical device according to claim 1, wherein the control unit is configured to include information or a value which indicates an oxygen saturation in the blood of the living being, and/or information or a value which indicates an expiratory carbon dioxide concentration in the exhaled gas of the living being, in the coordination of alarms.

10. A medical device according to claim 1, wherein the control unit is adapted to include information indicating a ratio of an oxygen saturation to an expiratory carbon dioxide concentration in the coordination of alarms.

11. A medical device with alarm coordination for ventilation of a living being, the medical device comprising:
a breathing gas supply system, which comprises a gas conveying unit, a dosing unit for controlled dosing of an inspiratory breathing gas quantity and a device for controlling an expiratory breathing gas quantity;
a sensor system, which comprises a flow rate sensor, which is in operative connection with the breathing gas supply system to continuously detect at least one measured value of a flow rate and to make the measured value available;
a line system, which is configured to supply quantities of breathing gas to the living being and to guide quantities of breathing gas away from the living being;
an alarm unit configured to issue an alarm; and
a control unit operatively connected to the sensor system to receive the measured values made available from the sensor system, operatively connected to the breathing gas supply system and operatively connected to the alarm unit,
wherein the control unit is configured to carry out and coordinate with the sensor system, the breathing gas supply system and the line system, a controlled dosing of the inspiratory breathing gas quantity for ventilation of the living being,
wherein the control unit is configured to determine a tidal volume based on the measured values of the flow rate sensors,
wherein the control unit is configured to carry out a coordination of alarms during a course of ventilation over a plurality of inspiratory dosing periods, the alarms being directed to undershoots of a lower volume threshold value of the tidal volume,
wherein the control unit is configured to determine a degree of undershoot as a criterion for the coordination of alarms, which degree of undershoot indicates a degree to which a tidal volume falls below the lower volume threshold value,
wherein the control unit is configured to carry out an adjustment of an alarm delay based on the degree of undershoot; and
wherein the control unit is configured to control the alarm unit such that the alarm unit issues an alarm based on the adjusted alarm delay.

12. A medical device according to claim 11, wherein the control unit is configured to determine a frequency indicator as a further criterion for the coordination of alarms, wherein the frequency indicator indicates a frequency with which tidal volume falls below the lower volume threshold value during a predetermined number of inspiratory dosages and the control unit is configured to carry out the adjustment of the alarm delay based also on the frequency indicator.

13. A medical device according to claim 11, wherein the control unit is configured to carry out an averaging, mean value calculations or filtering over tidal volumes over at least two inspiratory phases to form a value used for determining the degree of undershoot.

14. A medical device according to claim 12, wherein the control unit is configured to determine the degree of undershoot and/or the frequency indicator based on an averaging or a mean value calculation or a method of linear interpolation or a method of non-linear interpolation.

15. A medical device according to claim 14, wherein the control unit is configured to implement an upper threshold of the determined current tidal volume before or during the averaging and/or interpolation over the tidal volume.

16. A medical device according to claim 12, further comprising a data memory that is operatively connected with the control unit, wherein the data memory is configured to store, save and make available to the control unit the degree of undershoot and/or the frequency indicator as one or more of:
tables or tables with threshold values or tolerances;
data sets or tables with threshold values or tolerance bands;
data sets or tables with comparison values and/or comparison curves; and
data sets or tables with specifications and/or specification curves.

17. A medical device according to claim 11, wherein the control unit is configured to include a type of a ventilation mode used during the carrying out of ventilation as a further criterion for the coordination of alarms.

18. A medical device according to claim 11, wherein the control unit is configured to include patient-demographic criteria or patient-specific criteria as further criteria for the coordination of alarms.

19. A medical device according to claim 11, wherein the control unit is configured to include information or a value which indicates an oxygen saturation in the blood of the living being, and/or information or a value which indicates an expiratory carbon dioxide concentration in the exhaled gas of the living being as further criteria for the coordination of alarms.

20. A medical device according to claim 11, wherein the control unit is adapted to include information indicating a ratio of an oxygen saturation to an expiratory carbon dioxide concentration as a further criterion for the coordination of alarms.
